(19) United States
(12) Patent Application Publication
(10) Pub. No.: US 2024/0307618 A1
(43) Pub. Date: Sep. 19, 2024

(54) PREDICTION OF MEAL AND/OR EXERCISE EVENTS BASED ON PERSISTENT RESIDUALS

(71) Applicant: INSULET CORPORATION, Acton, MA (US)

(72) Inventors: Joon Bok LEE, Acton, MA (US); Yibin ZHENG, Hartland, WI (US); Jason O'CONNOR, Acton, MA (US)

(21) Appl. No.: 18/675,900

(22) Filed: May 28, 2024

Related U.S. Application Data
(63) Continuation of application No. 17/142,425, filed on Jan. 6, 2021, now Pat. No. 12,036,389.
(60) Provisional application No. 62/957,620, filed on Jan. 6, 2020.

(51) Int. Cl. A61M 5/172; A61M 5/142

(52) U.S. Cl. CPC A61M 5/1723; A61M 5/14244; A61M 2205/52; A61M 2230/201

(57) ABSTRACT

Exemplary embodiments provide an approach to predicting meal and/or exercise events for an insulin delivery system that otherwise does not otherwise identify such events. The insulin delivery system may use a model of glucose insulin interactions that projects estimated future glucose values based on the history of glucose values and insulin deliveries for the user. The predictions of meal events and/or exercise events may be based on residuals between actual glucose values and predicted glucose values. The exemplary embodiments may calculate a rate of change of the residuals over a period of time and compare the rate of change to thresholds to determine whether there likely has been a meal event or an exercise event. The insulin delivery system may then take measures to account for the meal or exercise by the user.

### CROSS-REFERENCE TO RELATED APPLICATIONS

This application is a continuation of U.S. application Ser. No. 17/142,425, filed Jan. 6, 2021, which claims the benefit of the filing date of U.S. Provisional Application Ser. No. 62/957,620, filed Jan. 6, 2020, the entire contents of which are incorporated herein by reference in their entirety.

### BACKGROUND

Patients with type 1 diabetes may be treated with insulin deliveries in a number of different ways. One approach is to manually deliver a correction bolus of insulin to patients as needed. For instance, if a patient's blood glucose level is 170 mg/dL and the target blood glucose level is 120 mg/dL, a bolus of 1 U may be manually delivered to the patient (assuming a correction factor of 1:50). There are some potential problems with manually delivering such boluses to the patient. The patients may deliver improper amounts of insulin in the bolus. For instance, the user may need a significantly lower amount of insulin than the bolus amount of 1 U. The insulin that has been delivered cannot be taken back from the patient's bloodstream. As a result, the delivery of the bolus may put the patient at risk of hypoglycemia.

Another approach is for the insulin to be delivered automatically by an insulin pump system. This approach may overcome some of the problems with manual delivery of insulin boluses. The insulin pump systems may use a closed loop control system for regulating the amount of insulin delivered at fixed intervals, such as every 5 minutes. The closed loop algorithms used by the control system may employ a penalty for large insulin deliveries that is balanced in a cost function with a penalty for glucose level excursions. The use of the cost function typically results in smaller insulin deliveries that are delivered more frequently than the manually delivered boluses. The closed loop system may reassess a patient's need more often than a manual approach. These systems, however, may be error prone and may not account for all relevant factors.

### SUMMARY

In accordance with an exemplary embodiment, a method is performed by a processor. Per the method, an actual blood glucose concentration history for a user is obtained. The actual blood glucose concentration history contains actual blood glucose concentration values and indications of when the actual blood glucose concentration values were obtained. A predicted blood glucose concentration history for the user is obtained. The predicted blood glucose concentration history contains predicted blood glucose concentration values and indications of when the predicted blood glucose concentration values were obtained. The predicted blood glucose concentration values in the blood glucose concentration history are generated using a model of glucose and insulin interactions. Residual values are calculated between values in the actual blood concentration history with like times in the predicted blood glucose concentration history over a time window. A rate of change of the residual values for groups of residual values for consecutive times in the time window is calculated. At least one calculated rate of change of the residual values for at least one of the groups is identified as having a magnitude that exceeds a threshold and that is positive. Based on the identifying, it is determined that the user has ingested a meal, and it is designating in the model that a meal was ingested by the user.

The method may further include delivering insulin to the user in response to designation of the meal event. The method may include delivering a bolus of insulin via drug delivery device. The delivering may comprise delivering a larger dosage of insulin during a basal insulin delivery. The threshold may be tailored to the insulin sensitivity of the user. The threshold may be set based on an empirical blood glucose response of the user to ingesting a mcal.

In accordance with an exemplary embodiment, a method is performed by a processor. An actual blood glucose concentration history for a user is obtained. The actual blood glucose concentration history contains actual blood glucose concentration values and indications of when the actual blood glucose concentration values were obtained. Predicted blood glucose concentration history for the user is obtained. The predicted blood glucose concentration history contains predicted blood glucose concentration values and indications of when the predicted blood glucose concentration values were obtained. The predicted blood glucose concentration values in the blood glucose concentration history are generated by a model of glucose and insulin interactions. Residual values are calculated between values in the actual blood concentration history with like times in the predicted blood glucose concentration history over a time window. A rate of change of the residual values for groups of residual values for consecutive time times in the time window is calculated. At least one calculated rate of change of the residual values for at least one of the groups is identified as having a magnitude that exceeds a negative threshold and that is negative. Based on the identifying, it is determined that the user has exercised, and an exercise event by the user is designated in the model.

The method may further entail suspending basal delivery of insulin to the user from a drug delivery device in response to the designation of an exercise event. The drug delivery device may be a wearable insulin pump. The method may further entail reducing a basal delivery dosage of insulin from a drug delivery device in response to the designation of an exercise event. The threshold may be tailored to the user. The threshold may be based on empirical blood glucose concentration response of the user to exercise.

In accordance with an exemplary embodiment, a device for controlling delivery of insulin to a user via a drug delivery device includes a storage for storing an actual blood glucose concentration history for a user, a predicted blood glucose concentration history for the user, a model of glucose insulin interactions for the user and a control application for controlling a drug delivery device for delivering insulin to the user. The actual blood glucose concentration history contains actual blood glucose concentration values and indications of when the actual blood glucose concentration values were obtained, and the predicted blood glucose concentration history contains predicted blood glucose concentration values and indications of when the predicted blood glucose concentration values were obtained. The predicted blood glucose concentration values in the blood glucose concentration history are generated by the model of glucose and insulin interactions. The device also includes a processor for executing instructions causing the processor to calculate residual values between values in the actual blood concentration history with like times in the predicted blood glucose concentration history over a time window. The instructions also cause the processor to calculate a rate of change of the residual values for groups of residual values for consecutive time times in the time window and identify at least one calculated rate of change of the residual values for at least one of the groups that has a magnitude that exceeds a positive threshold and that is positive or identify at least one calculated rate of change of the residual values for at least one of the groups that has a magnitude that exceeds a negative threshold and that is negative. Where it is identified that at least one calculated rate of change of the residual values for at least one of the groups has a magnitude that exceeds the positive threshold and is positive, it is determined that the user has ingested a meal, and a meal event by the user is designated in the model. Where it is identified that at least one calculated rate of change of the residual values for at least one of the groups has a magnitude that exceeds the negative threshold and is negative, it is determined that the user has exercised, and an exercise event by the user is designated in the model.

The processor may also cause remedial measures to be taken by the drug delivery device in response to designating a meal event or an exercise event. The remedial measures may comprise at least one of delivering a bolus of insulin to the user, increasing dosage of a basal insulin delivered to the user, suspending delivery of insulin to the user or decreasing dosage of a basal insulin delivered to the user. At least one of the positive threshold or the negative threshold may be customized to the user. The device may be an insulin pump device or the device may be a separate device that controls an insulin pump device.

### BRIEF DESCRIPTION OF THE DRAWINGS

FIG. 1 depicts a block diagram of an illustrative drug delivery system suitable for practicing an exemplary embodiment.

FIG. 2 depicts contents of an illustrative storage for use in the drug delivery system.

FIG. 3 illustrates a block diagram showing inputs and output of a glucose insulin interaction model.

FIG. 4 depicts a flowchart showing illustrative steps for determining residuals between actual blood glucose concentration values and predicted blood glucose concentration values.

FIG. 5 depicts a flowchart showing illustrative steps for determining a rate of change of residual values for a cycle.

FIG. 6A depicts a flowchart showing illustrative steps for determining when to designate a meal event or an exercise event.

FIG. 6B depicts a flowchart showing illustrative steps for determining when to designate different types of meal events and different types of exercise events.

FIG. 6C depicts a flowchart showing illustrative steps that may be performed when both magnitude of a residual and rate of change of residuals are used to determine whether to designate a meal event type or an exercise event type.

FIG. 7 depicts a flowchart showing illustrative steps for responding to a meal event.

FIG. 8 depicts a flowchart showing illustrative steps for responding to an exercise event.

### DETAILED DESCRIPTION

The exemplary embodiments address some of the limitations of some conventional insulin delivery system control systems. Exemplary embodiments provide an approach to predicting meal and/or exercise events for an insulin delivery system that otherwise does not otherwise identify such events. The insulin delivery system may use a model of glucose insulin interactions that projects estimated future glucose values based on the history of glucose values and insulin deliveries for the user. The predictions of meal events and/or exercise events may be based on residuals between actual glucose values and predicted glucose values. The exemplary embodiments may calculate a rate of change of the residuals over a period of time (such as over a fifteen minute period) and compare the rate of change to thresholds to determine whether there likely has been a meal event or an exercise event. For instance, if the rate of change is positive and the rate is above a first threshold, it is indicative of a meal event, and such a meal event may be designated. On the other hand, if the rate of change is negative and the rate of change is below a second threshold, it is indicative of an exercise event, and such an event may be designated. The drug delivery system may then take measures to account for the meal or exercise by the user.

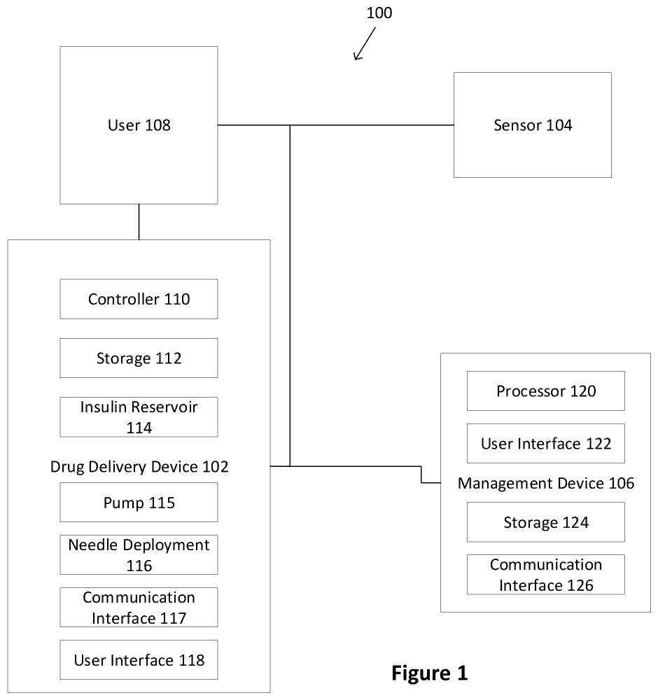

FIG. 1 depicts an illustrative drug delivery system 100 that is suitable for delivering insulin to the user 108 in an exemplary embodiment. The drug delivery system 100 includes a drug delivery device 102. The drug delivery device 102 may be a wearable device that is worn on the body of the user 108. The drug delivery device 102 may be directly coupled to the user 108 (e.g., directly attached to a body part and/or skin of the user 108 via an adhesive or the like). In an example, a surface of the drug delivery device 102 may include an adhesive to facilitate attachment to the user 108.

The drug delivery device 102 may include a controller 110. The controller 110 may be implemented in hardware, software, or any combination thereof. The controller 110 may, for example, be a microprocessor, a logic circuit, a field programmable gate array (FPGA), an application specific integrated circuit (ASIC) or a microcontroller coupled to a memory. The controller 110 may maintain a date and time as well as other functions (e.g., calculations or the like). The controller 110 may be operable to execute an algorithm stored in the storage 112 that enables the controller 110 to direct operation of the drug delivery device 102. In addition, the controller 110 may be operable to receive data or information. The storage 112 may include both primary memory and secondary memory. The storage 112 may include random access memory (RAM), read only memory (ROM), optical storage, magnetic storage, removable storage media, solid state storage or the like.

The drug delivery device 102 may include an insulin reservoir 114 for storing insulin for delivery to the user 108 as warranted. A pump 115 may be provided for pumping the insulin out of the insulin reservoir 114 to the user 108. A needle deployment component 116 may be provided to control deployment of needles or cannulas from the drug delivery device 102 to the user 108. The needle deployment component 116 may, for example, include a needle, a cannula and/or any other fluid path components for coupling the stored liquid drug in the insulin reservoir 114 to the user 108. The cannula may form a portion of the fluid path component coupling the user to the insulin reservoir 114. After the needle deployment component 116 has been activated, a fluid path to the user is provided, and the pump 115 may expel the liquid drug from the reservoir 114 to deliver the liquid drug to the user via the fluid path. The fluid path may, for example, include tubing coupling the drug delivery device 102 to the user 108 (e.g., tubing coupling the cannula to the reservoir 114).

The communications interface 117 may provide a communications link to one or more management devices physically separated from the drug delivery device 102 including, for example, a management device 104 of the user and/or a caregiver of the user. The communication link provided by the communications interface 117 may include any wired or wireless communication link operating according to any known communications protocol or standard, such as Bluetooth®, Wi-Fi, a near-field communication standard, a cellular standard, or any other wireless protocol The drug delivery device 102 may also include a user interface 118, such as an integrated display device for displaying information to the user 108 and in some embodiments, receiving information from the user 108. The user interface 118 may include a touchscreen and/or one or more input devices, such as buttons, knob or a keyboard.

The drug delivery system 100 may include a sensor 104 for sensing the blood glucose concentration levels of the user 108. The sensor 104 may be a glucose monitor that provides periodic blood glucose concentration measurements, such as a continuous glucose monitor (CGM), or another type of device or sensor that provides blood glucose measurements. The sensor 104 may be physically separate from the drug delivery device 102 or may be an integrated component thereof. The sensor 104 may provide the controller 110 with data indicative of measured or detected blood glucose levels of the user 108. The sensor 104 may be coupled to the user by, for example, adhesive or the like and may provide information or data on one or more medical conditions and/or physical attributes of the user 108. The information or data provided by the sensor 104 may be used to adjust drug delivery operations of the drug delivery device 102.

The drug delivery system 100 may also include the management device 106. The management device 106 may be a special purpose device, such as a personal diabetes manager (PDM). The management device 106 may be a programmed general purpose device, such as any portable electronic device including, for example, a dedicated controller, such as processor, a smartphone, or a tablet. The management device 106 may be used to program or adjust operation of the drug delivery device 102 and/or the sensor 104. The management device 106 may be any portable electronic device including, for example, a dedicated controller, a smartphone, or a tablet. In the depicted example, the management device 106 may include a processor 120, a storage 124, and a communication interface 126. Processor 120 may execute processes to manage a user's blood glucose levels and for control the delivery of the drug or therapeutic agent to the user 108. The processor 120 may also be operable to execute programming code stored in the management storage 124. For example, the storage may be operable to store one or more control applications for execution by the processor 120. The communication interface 126 may include a receiver, a transmitter, or a transceiver that operates according to one or more radio-frequency protocols. For example, the communication interface 126 may include a cellular transceiver and a Bluetooth transceiver that enables the management device 106 to communicate with a data network via the cellular transceiver and with the sensor 104 and the drug delivery device 102. The respective transceivers of communication interface 126 may be operable to transmit signals containing information useable by or generated by an application or the like. The communication interfaces 117 and 126 of respective wearable drug delivery device 102 and sensor 104, respectively, may also be operable to transmit signals containing information useable by or generated by an application or the like.

The management device 106 may include a user interface 122 for communicating with the user 108. The user interface 122 may include a display, such as a touchscreen, for displaying information. The touchscreen may also be used to receive input when it is a touch screen. The user interface 122 may also include input elements, such as a keyboard, button, knobs or the like.

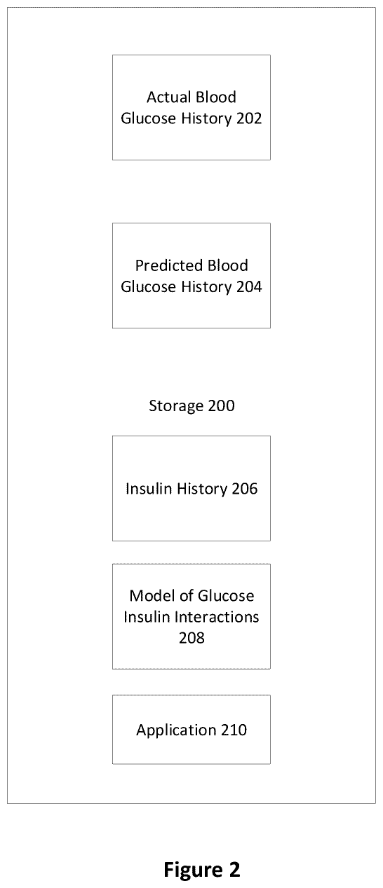

FIG. 2 shows an example of contents held in a storage 200, such as storage 112 of the drug delivery device or storage 124 of the management device 106. The storage 200 may hold the actual blood glucose history 202, which contains the history of blood glucose concentration readings from the sensor 104 for the user 108 over time. The storage 200 may hold the predicted blood glucose history containing the predicted blood glucose values for the user 108 generated by the model used in the drug delivery system. This model 108 may be encoded in application 210 that is executed by the controller 110 with input from the management device 106 and sensor 104. The application 210 may be executed to control the drug delivery device 102 and oversee activities of the drug delivery device 102. The storage 200 may also store the insulin history 206 that records the insulin deliveries and delivery times and/or cycles for the user 108.

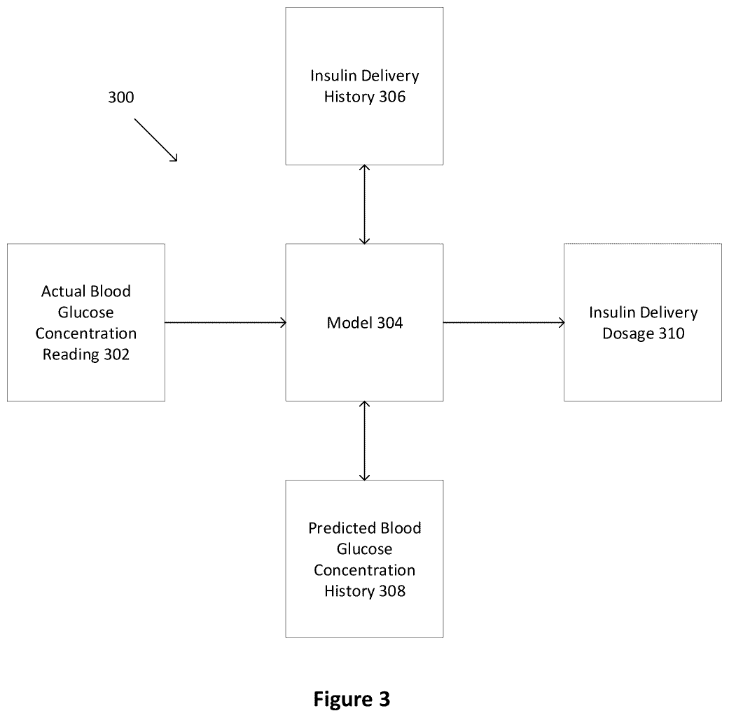

FIG. 3 depicts a block diagram 300 showing input and outputs of the model 304 of glucose/insulin interactions that is used by the application 210 in managing the drug delivery device 102. The model 304 receives the actual blood glucose concentration reading 302 from the sensor 104. The actual blood glucose concentration readings may be delivered at periodic intervals, such as cycles of every 5 minutes, in some embodiments. The actual blood glucose concentration in compared to the predicted blood glucose concentration for the same time in the predicted blood glucose concentration history 308. The model 304 predicts blood glucose concentration at times. The predictions are used to determine and control the system response to actual blood glucose concentrations. The predicted blood glucose concentration history 308 holds the values predicted by the model 304 over time. The model 304 also looks at the insulin delivery history 306 for the user. The insulin delivery history 306 contains the dosages and times and/or cycles of all bolus insulin deliveries and basal insulin deliveries to the user. Based on the inputs 302, 306 and 308, the model 304 generates an output control signal to the pump 115 that specifies an insulin delivery dosage 310 if warranted. The dosage may be zero, in which case no insulin is to be delivered. The model 304 may seek to deliver small dosages rather than larger boluses to avoid hypoglycemic or hyperglycemic risks. Moreover, the model 304 may account for other parameters that affect blood glucose concentration levels.

As was mentioned above, the exemplary embodiments embellish the model 304 to account for meals by the user and exercise by the user. The exemplary embodiments may identify meals or exercise by looking at residuals between the actual blood glucose concentration values for the user and predicted blood glucose concentration values. The difference between the actual values and the predicted values provide a magnitude of error for the predictions. Such error may come from multiple sources. In order to identify the error as originating from an unaccounted meal or exercise, the exemplary embodiments may look for significant enough residuals that change substantially between successive readings in a time interval. For example, when a user ingests a meal, the blood glucose concentration for the user will increase fairly rapidly after ingestion and will continue to increase as the remaining portions of the meal are digested. The predicted blood glucose level will not anticipate such an increase in blood glucose concentration. Thus, a rapid increase in the residuals is indicative of the user ingesting a meal. When a user exercises, the blood glucose concentration level will drop fairly rapidly until the user stops exercising. The predicted blood glucose level will not anticipate such a decrease in blood glucose concentration level. Hence, the rate of change of the residuals also may be indicative of exercise in some instances.

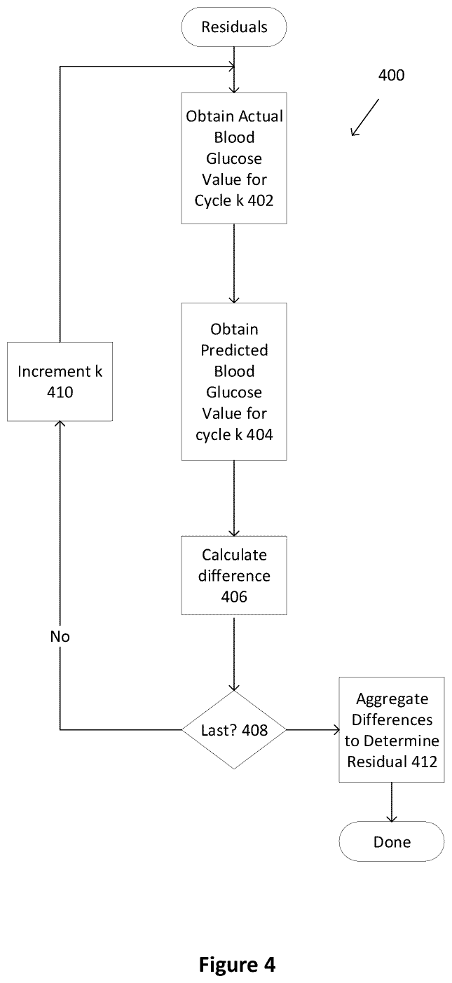

FIG. 4 depicts a flowchart 400 shows illustrative steps for determining residuals over time. First, the actual blood glucose concentration level for cycle k is determined (402), where k is a positive integer that serves as a cycle index value. The drug delivery system 100 may be configured to operate in periodic cycles, such as at 5 minute intervals, where the newest actual blood glucose concentration value is obtained and insulin deliveries may be made based on the latest value. This may be the value provided by the sensor 104 at cycle k. The predicted blood glucose concentration level at cycle k is also obtained (404). A recursive model of n^{th} order may be used to project the future glucose values Gₚ. The future glucose values are predicted from the past blood glucose concentration values and insulin delivery values as:

$${G}_{p} ( k ) = {b}_{1} G ( k - 1 ) + {b}_{2} G ( k - 2 ) + … {b}_{n} G ( k - n ) + I ( k - 1 ) + I ( k - 2 ) + … I ( k - n )$$

Where Gₚ(k) is the predicted glucose value at cycle k; G(k) is an actual blood glucose concentration value at cycle k; bₓ is a weight assigned to the past coefficient where x is an index value ranging from 1 to n; and I(k) is an insulin action for a dosage amount (i.e., how much the dosage of insulin will reduce the blood glucose concentration) delivered at cycle k.

The difference for cycle k may be calculated by subtracting the predicted blood glucose concentration value from the actual blood glucose concentration value (406). If this is the last cycle of interest (see 408), then the process is complete. If not, the cycle index is incremented (410) and the process is repeated beginning at (402).

The prediction residual for each cycle may be calculated by comparing the actual blood glucose concentration values to the predicted blood glucose concentration values over cycles k to k+m and summing those differences (412) as follows:

$$R ( k + m ) = {\sum }_{q = 0}^{m} G ( k + q ) - {G}_{p} ( k + q )$$

Where R(k+m) is the residual for the cycle k+m; and q is an index ranging from 0 to m.

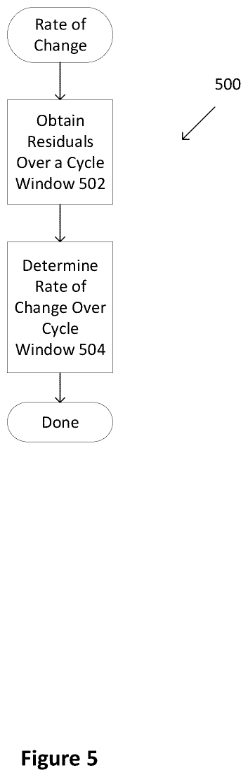

As was mentioned above, it is not the residuals for successive cycles alone that are or interest but rather the rate of change of the residuals over multiple successive cycles that is of interest. FIG. 5 shows a flowchart 500 of how the rate of change of the residuals is determined. Initially, the residuals over a window of successive cycles are obtained (502). In one exemplary case, the residuals for 3 successive cycles may be obtained in an exemplary case. The residuals for each cycle may be calculated using the summation set forth above. The rate of change of the residuals is then determined (504). This may be calculated by calculating the difference between the first and last residuals in a group of three successive cycles and dividing the sum by the number of cycles. If three cycles constitute the time window of instance, the rate of change may be expressed as:

$${ROC}_{3} ( k + m ) = \frac{R ( k + m ) - R ( k + m - 2 )}{3}$$

Where ROC₃(k 30 m) is the rate of change of the residual over three successive cycles spanning cycles k through m and R(i) is the residual for cycle i.

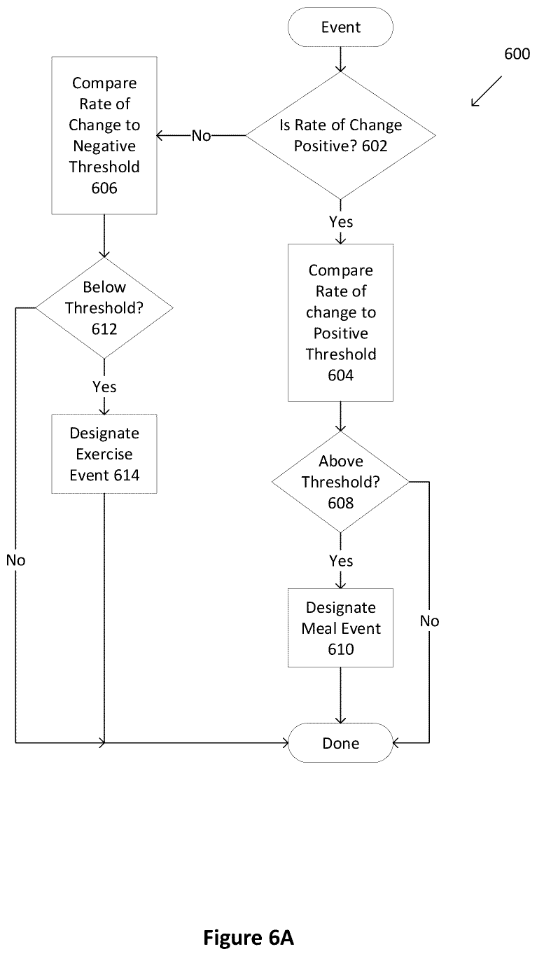

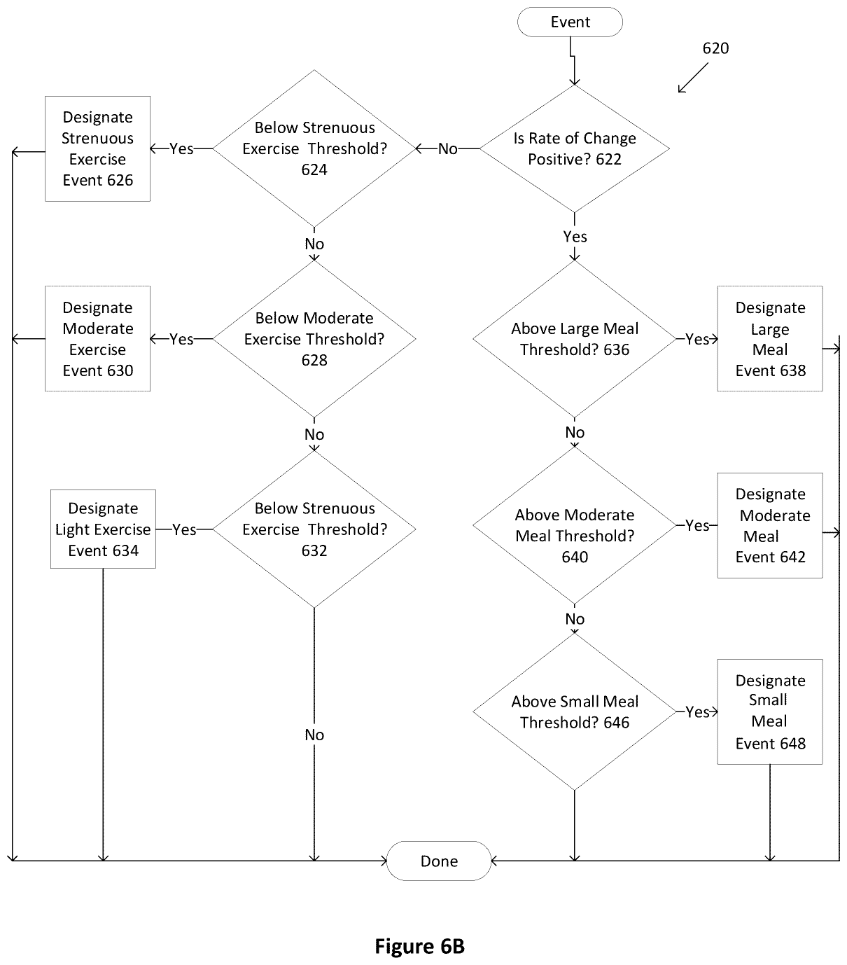

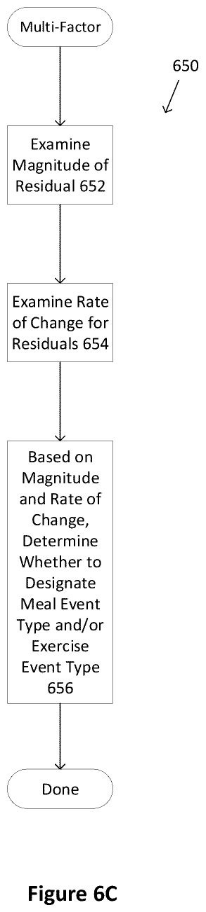

The rates of change of the residuals may then be analyzed to identify meal events or exercise events. FIG. 6A depicts a flowchart 600 showing illustrative steps for determining such events. A check may be made whether the rate of change of the residuals in positive (602). If the rate of change is positive, it implies that the actual blood glucose concentration values are increasing more rapidly than the predicted blood glucose concentration values. The rate of change is compared to a positive threshold (604). The comparison to the positive threshold helps to rule out rates of change that are not as substantial as would be evidenced by a meal. If the rate of change is above the positive threshold (608), a meal event is designated (610). If the rate of change is not positive (see 602), the rate of change may be compared with a negative threshold (606). If the rate of change is below the negative threshold (i.e., more negative than the threshold) (612), an exercise event may be designated (614). Otherwise, no event is designated.

The positive threshold for determining meal events and the negative threshold for determining exercise events may be customized for the user. For example, data may be gathered for the user for actual meal events and the residuals and residual rate of change may be calculated for those actual meal events. Based on the actual data, the positive threshold may be set to distinguish meal events from other phenomena. Similarly, empirical data regarding actual exercise events concerning the residuals and the rate of change of the residuals for actual exercise events may be used to set the negative threshold.

Multiple thresholds can also be generated to define different degrees or types of meal events and exercise events. For example, the magnitudes of residuals and residual rate of change can be varying in tandem or independently depending on the type of a meal, where a slow absorbing meal may exhibit a slower increase in glucose and thus a smaller residual versus a fast-absorbing meal. A larger meal of the same type may simply exhibit a similar error for a longer period, and the residual threshold can be set higher if the goal is to detect larger meals only. Similarly, a fast-absorbing meal may exhibit a temporarily rapid increase in glucose, and thus the residual rate of change threshold can also be set higher to detect such a meal. Similarly, aerobic and anaerobic exercises can exhibit different patterns of residuals versus predictions. Short, intense bouts of exercise may indicate high rates of change but not a significant increase in thresholds, while leisurely, longer acting activities may result in lower rates of change but persistent increases over time. Therefore, the combination of residual thresholds and residual rate of change thresholds can be tuned differently, or tuned for multiple instances, to allow for detection of both event types and different degrees of meals and exercise.

FIG. 6B depicts a flowchart showing illustrative steps that may be performed to designate different types of meal events and/or different types of exercise events for an illustrative case. In this instance, the rate of change of the residual dictates the designation and categorization of exercise and meal events. First, a check in made whether the rate of change of the residuals in positive or negative (622). If the rate of change is negative, it may be an indication that the user is exercising. Hence, a check is made whether the rate of change is below (i.e., more negative) than a strenuous exercise threshold (624). If the rate of change is below the strenuous exercise threshold, a strenuous exercise event is designated (626). If not, a check is made whether the rate of change is below a moderate exercise threshold (628). If the rate of change is below the moderate exercise threshold, a moderate exercise threshold event is designated (630). If the rate of change is not below the moderate exercise threshold, a check is made whether the rate of change is below a light exercise threshold (632). If the rate of change is below the light exercise threshold, a light exercise event is designated (634). Otherwise, no exercise event is designated.

If it is determined that the rate of change is positive (see 622), it may be an indication that the user has eaten. A check is made whether the rate of change is greater than a large meal threshold (636). If so, a large meal event is designated (638). If not, a check is made whether the rate of change is above a moderate meal threshold (640). If so, a moderate meal event is designated (642). If not, a check is made whether the rate of change is above a small meal threshold (646). If so, a small meal event is designated (648). If not, no meal event is designated.

In another variant, as mentioned above, both the magnitude of the residual and the rate of change of the residual may be used to determine meal or exercise event designations of different degrees. FIG. 6C depicts a flowchart (650) of illustrative steps that may be performed for this variant. The process may entail examining the magnitude of the residual (e.g., the current residual) (652). The rate of change of the residuals (e.g., the latest rate of change) is examined as well (654). Based on both the magnitude of the residual and the rate of change of the residual, a determination is made whether to designate a meal event of a given degree or an exercise event type of a given degree (656). As mentioned above, combinations of thresholds may be used to make this determination and cause a designation as warranted.

Another approach is to relate a user's total insulin delivery (TDI) for a day and relate it to the user's insulin to carbohydrate ratio that identifies how much insulin is required to offset a specified amount of carbohydrates. These values may be related by heuristic rules, such as the 800 rule, which looks at 800/TDI to determine the ratio of a carbohydrate ingestion amount that may be offset by 1 unit of insulin. The correction factor may specify how much of a drop in glucose is realized by one unit of insulin, through heuristics such as the 1800 rule (1800/TDI). A combination of these rules can be utilized to estimate the quantity and presence of the user's events. For instance, it may be desired that the system would detect a meal event with carbohydrate quantity above a certain threshold, such as 30 grams. These 30 grams of carbohydrates can be converted into the estimated amount of insulin that would be needed to compensate for the event. If the user's TDI is 50 U, then the 800 rule (800/TDI) means 1 U of insulin would compensate for 16 g, and the 30 grams of carbohydrates require 1.875 U of insulin. This can also be correlated to expected glucose rise, based on the user's correction factor, which through the 1800 rule (1800/TDI) would mean 1 U of insulin reduces glucose by 36 mg/dL, and 1.875 U of insulin would reduce insulin by 67.5 mg/dL. Therefore, the threshold can be designed in such a way that an unexpected rise in glucose rise by more than 67.5 mg/dL would be detected.

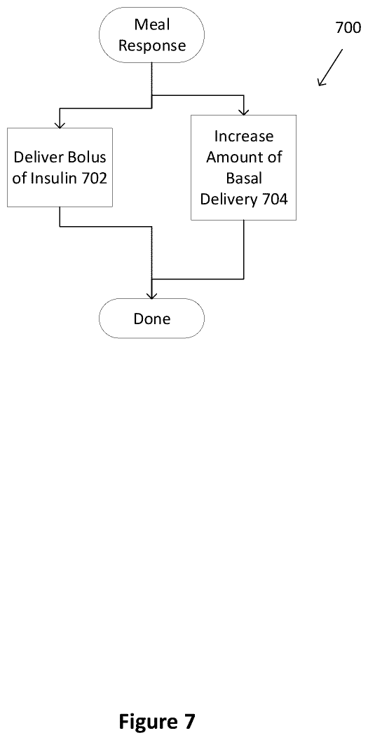

The drug delivery system 100 may respond to the designation of a meal event and/or an exercise event. FIG. 7 depicts a flowchart 700 showing illustrative steps that may be performed in response a meal event being designated. Since, a meal event indicates that the user has ingested food that has raised the actual blood glucose concentration level of the user, the drug delivery system 100 may take steps to reduce the blood glucose concentration level of the user. For instance, a bolus of insulin may be delivered to the user (702). Another option is to increase the dosage of the basal insulin delivery (704). These options may be done automatically by the controller 110 which is executing the application for controlling the drug delivery device 102. The steps 702 and 704 may be realized by loosening constraints in the model to allow greater insulin delivery.

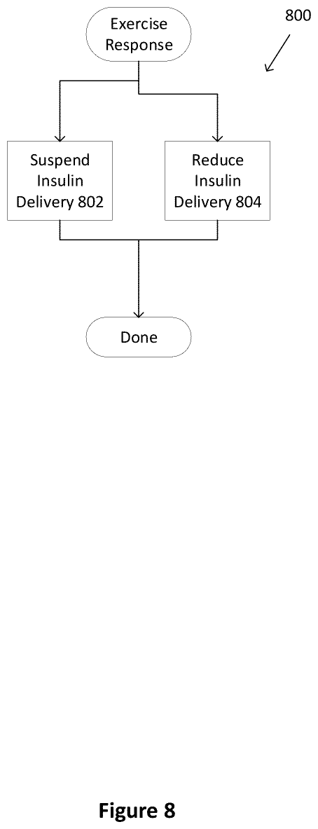

FIG. 8 depicts a flowchart 800 showing illustrative steps that may performed in response to the designation of an exercise event. Exercise will reduce the blood glucose concentration level of the user. Thus, the aim is to take steps to avoid further decreases in the blood glucose concentration level of the user. One option is to halt deliveries of basal insulin to the user for a specified period of time (802). Another option is to decrease the dosage level of the automatically delivered insulin (804). These options may be done automatically by the controller 110 which is executing the application for controlling the drug delivery device 102. The constraints in the model may be adjusted to suspend or decrease the insulin delivery in (802) and (804).

In an alternative embodiment, the values of the residuals are not used; rather the summed square of the residuals are used. The summed square of residuals R_{SSR} can be expressed as an extension to the equation in 0031 where

$${R}_{SSR} ( k + m ) = {\sum }_{q = 0}^{m} {( G ( k + q ) - {G}_{p} ( k + q ) )}^{2}$$

This helps to reduce the effect of outliers using the previously described approach. The signs of the residuals must be maintained as the squaring will make all the squares positive. One option is to set all of the residuals of an undesired sign (i.e., the sign not of interest when comparing to a particular threshold) to zero.

While the present invention has been described with reference to exemplary embodiments thereof, it should be appreciated that various changes in form and/or detail may be made without departing from the intended scope of the present invention as defined in the appended claims. For example, the residuals may also be used to identify other unknown system events, such as error produced by pressure-induced sensor attenuation. Pressure-induced sensor attenuations may result in sudden reduction in glucose concentrations similar to immediate glucose outcomes following exercise events, and may be detected in a similar manner by observing sudden unexpected accumulation of negative residual values versus predictions.

1. A drug delivery system, comprising;
a drug delivery device for delivering insulin to the user;
a storage for storing an actual glucose history for a user, a predicted glucose history for the user, a model of glucose insulin interactions for the user and a control application for controlling a drug delivery device for delivering insulin to the user;
wherein the actual glucose history contains actual glucose values and indications of when the actual glucose values were obtained and the predicted glucose history contains predicted glucose values and indications of when the predicted glucose values were obtained and wherein the predicted glucose values in the glucose history are generated by the model of glucose and insulin interactions;
a processor for executing instructions causing the processor to:
calculate residual values between glucose values in the actual glucose history with like times in the predicted glucose history over a time window;
calculate a rate of change of the residual values for groups of residual values for consecutive times in the time window;
based on the calculated rate of change of the residual values for the groups of residual glucose values, determining that the user has either ingested a meal or exercised.

2. The drug delivery system of claim 1, wherein the processor further executes instructions that cause the processor to:
identify at least one calculated rate of change of the residual values for at least one of the groups that has a magnitude that exceeds a positive threshold and that is positive or identify at least one calculated rate of change of the residual values for at least one of the groups that has a magnitude that exceeds a negative threshold and that is negative; and
responsive to the identifying, take a remedial measure that affects delivery of insulin delivered to the user.

3. The drug delivery system of claim 2, wherein further responsive to the identifying, where it is identified that at least one calculated rate of change of the residual values for at least one of the groups has a magnitude that exceeds the positive threshold and is positive, the processor determines that the user has ingested a meal, designates a meal event by the user in the model, and performs the remedial measure to compensate for an increase in glucose level of the user due to the meal event.

4. The drug delivery system of claim 3, wherein the remedial measure comprises increasing dosage of a basal insulin delivered to the user or delivering a bolus of insulin to the user.

5. The drug delivery system of claim 2, wherein further responsive to the identifying, where it is identified that at least one calculated rate of change of the residual values for at least one of the groups has a magnitude that exceeds the negative threshold and is negative, the processor determines that the user has exercised, designates an exercise event by the user in the model, and performs the remedial measure to compensate for an decrease in glucose level of the user due to the exercise event.

6. The drug delivery system of claim 5, wherein the remedial measure comprises decreasing dosage of a basal insulin delivered to the user or suspending delivery of basal insulin to the user.

7. The drug delivery system of claim 2, wherein at least one of the positive threshold or the negative threshold is customized to the user.

8. The drug delivery system of claim 1, wherein the drug delivery device is an insulin pump device.

9. The drug delivery system of claim 1, wherein the storage and the processor are part of a separate device that controls the drug delivery device.

10. A method performed by a processor in a drug delivery system, comprising:
with the processor, calculate residual values between glucose values in an actual glucose history with like times in a predicted glucose history over a time window;
with the processor, calculate a rate of change of the residual values for groups of residual values for consecutive times in the time window;
with the processor, based on the calculated rate of change of the residual values for the groups of residual values, determining that the user has either ingested a meal or exercised.

11. The method of claim 10, further comprising:
with the processor, identifying at least one calculated rate of change of the residual values for at least one of the groups that has a magnitude that exceeds a positive threshold and that is positive or identify at least one calculated rate of change of the residual values for at least one of the groups that has a magnitude that exceeds a negative threshold and that is negative; and
with the processor, responsive to the identifying, taking a remedial measure that affects delivery of insulin delivered to the user.

12. The method of claim 11, wherein further responsive to the identifying, where it is identified that at least one calculated rate of change of the residual values for at least one of the groups has a magnitude that exceeds the positive threshold and is positive, determining that the user has ingested a meal, designating a meal event by the user in the model, and performing the remedial measure to compensate for an increase in glucose level of the user due to the meal event.

13. The method of claim 12, wherein the remedial measure comprises increasing dosage of a basal insulin delivered to the user or delivering a bolus of insulin to the user.

14. The method of claim 11, wherein further responsive to the identifying, where it is identified that at least one calculated rate of change of the residual values for at least one of the groups has a magnitude that exceeds the negative threshold and is negative, determining that the user has exercised, designating an exercise event by the user in the model, and performing the remedial measure to compensate for an decrease in glucose level of the user due to the exercise event.

15. The method of claim 14, wherein the remedial measure comprises decreasing dosage of a basal insulin delivered to the user or suspending delivery of basal insulin to the user.

16. The method of claim 11, wherein at least one of the positive threshold or the negative threshold is customized to the user.

17. A drug delivery system, comprising:
a drug delivery device for delivering a drug to a user;
a storage for storing computer programming instructions;
a processor configured to execute the computer programming instructions, the executing of the computer programming instructions causing the processor to:
determining deviations over a prediction interval between predictions of glucose levels of the user at times and actual glucose levels of the users at the times;
determining a rate of change among deviations over successive times in the prediction interval; and
based on the rate of change, determining that the user has exercised.

18. The drug delivery system of claim 17, wherein the executing of the computer programming instructions further causes the processor to modify delivery of the drug to the user by the drug delivery device.

19. The drug delivery system of claim 17, each of the times is associated with a cycle of the drug delivery system.

20. The drug delivery system of claim 18, wherein the determining that the user has exercised entails determining that the rate of change is negative and is more negative than a threshold value.
